(19) United States
(12) Patent Application Publication
(10) Pub. No.: US 2003/0187006 A1
(43) Pub. Date: Oct. 2, 2003

(54) Method for enhancing cognitive function

(76) Inventor: James Hagan, Harlow (GB)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/275,853

(22) PCT Filed: May 15, 2001
(86) PCT No.: PCT/GB01/02134

(30) Foreign Application Priority Data
May 16, 2000 (GB) 0011802.6

(51) Int. Cl. A61K 31/505; A61K 31/277

(52) U.S. Cl. 514/275; 514/521

(57) ABSTRACT

A method for enhancing cognitive function by administering to a patient in need thereof an effective amount of a PDE4 inhibitor.

### SCOPE OF THE INVENTION

This invention relates to a method for enhancing cognitive function by administering a PDE4 inhibitor as defined herein below.

### BACKGROUND OF THE INVENTION

This invention also relates to a method of mediating or inhibiting the enzymatic activity (or catalytic activity) of PDE 4 in a mammal and thereby enhancing cognition.

Phosphodiesterase 4 inhibitors are useful in the treatment of a variety of allergic and inflammatory diseases including: asthma, chronic bronchitis, atopic dermatitis, urticaria, allergic rhinitis, allergic conjunctivitis, vernal conjunctivitis, eosinophilic granuloma, psoriasis, rheumatoid arthritis, septic shock, ulcerative colitis, Crohn's disease, reperfusion injury of the myocardium and brain, chronic glomerulonephritis, endotoxic shock and adult respiratory distress syndrome. In addition, PDE IV inhibitors are useful in the treatment of diabetes insipidus, [idney Int., 37:362, 1990; Kidney Int., 35:494, 1989] and central nervous system disorders such as depression and multi-infarct dementia.

It has now been found that certain of these PDE 4 inhibitors can be used to increase learning, retention and/or recall, collectively called enhancing cognitive function.

### SUMMARY OF THE INVENTION

This invention relates to a method for enhancing cognitive function by administering to a patient in need thereof an effective amount of a PDE4 inhibitor which has an IC₅₀ ratio of about 0.1 or greater as regards the IC₅₀ for the PDE4 catalytic form which binds rolipram with a high affinity divided by the IC₅₀ for the form which binds rolipram with a low affinity.

In a further aspect there is provided a use of a PDE4 inhibitor which has an IC₅₀ ratio of about 0.1 or greater as regards the IC₅₀ for the PDE IV catalytic form which binds rolipram with a high affinity divided by the IC₅₀ for the form which binds rolipram with a low affinity for the manufacture of a medicament for enhancing cognitive function.

### PREFERRED EMBODIMENTS AND EXAMPLES

The PDE4-specific inhibitor used to practice the disclosed method may be any one which is known to inhibit the PDE4 enzyme or which is discovered to act in as PDE4 inhibitor, and which are only PDE4 inhibitors, not compounds which inhibit other members of the PDE family as well as PDE4. Generally it is preferred to use a PDE4 antagonists which has an IC₅₀ ratio of about 0.1 or greater as regards the IC₅₀ for the PDE4 catalytic form which binds rolipram with a high affinity divided by the IC₅₀ for the form which binds rolipram with a low affinity.

For purposes of this disclosure, the cAMP catalytic site which binds R and S rolipram with a low affinity is denominated the “low affinity” binding site (LPDE 4) and the other form of this catalytic site which binds rolipram with a high affinity is denominated the “high affinity” binding site (BPDE4). This term “HPDE4” should not be confused with the term “hPDE4” which is used to denote human PDE4.

The assay for identifying PDE4 inhibitors, which can be used to enhance cognition, is detailed in the Examples recited below.

It is now known that there are at least two binding forms on human monocyte recombinant PDE 4 (hPDE 4) with which inhibitors interact. One explanation for these observations is that HPDE 4 exists in two distinct forms. One binds the likes of rolipram and denbufylline with a high affinity while the other binds these compounds with a low affinity. The preferred PDE4 inhibitors of use in this invention will be those compounds which have a salutary therapeutic ratio, i.e., compounds which preferentially inhibit cAMP catalytic activity where the enzyme is in the form that binds rolipram with a low affinity, thereby reducing the side effects which apparently are linked to inhibiting the form which binds rolipram with a high affinity. Another way to state this is that the preferred compounds will have an IC₅₀ ratio of about 0.1 or greater as regards the IC₅₀ for the PDE4 catalytic form which binds rolipram with a high affinity divided by the IC₅₀ for the form which binds rolipram with a low affinity.

A further refinement of this standard is that of one wherein the PDE4 inhibitor has an IC₅₀ ratio of about 0.1 or greater; said ratio being the ratio of the IC₅₀ value for competing with the binding of 1 nM of [3H]R-rolipram to a form of PDE 4 which binds rolipram with a high affinity over the IC₅₀ value for inhibiting the PDE4 catalytic activity of a form which binds rolipram with a low affinity using 1 microM[3 ]M-cAMP as the substrate. A further explanation of this test can be found in U.S. Pat. No. 5,998,428 or PCT application PCT/US00/05363 which has the U.S. designated on the Request form, the text of both being incorporated herein by reference to the extent that that text is necessary to the practice of this invention.

Most preferred are those PDE4 inhibitors which have an IC₅₀ ratio of greater than 0.5, and particularly those compounds having a ratio of greater than 1.0.

One preferred group of compounds that can be used in this method is that of formula 1 or a solvate, hydrate or polymorph thereof, either alone or combined with a pharmaceutically acceptable excipient, wherein Formula (1) comprises:

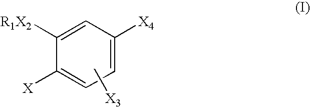

wherein:

R₁ is —(CR₄R₅)ₙC(O)O(CR₄R₅)ₘR₆, —(CR₄R₅)ₙC(O)NR₄(CR₄R₅)ₘR₆, —(CR₄R₅)ₙO(CR₄R₅)ₘR₆, or —(CR₄R₅)ᵣR₆ wherein the alkyl moieties may be optionally substituted with one or more halogens;

m is 0 to 2;

n is 1 to 4;

r is 0 to 6;

R₄ and R₅ are independently selected from hydrogen or a C₁₋₂ alkyl;

R₆ is hydrogen, methyl, hydroxyl, aryl, halo substituted aryl, aryloxyC₁₋₃ alkyl, halo substituted aryloxyC₁₋₃ alkyl, indanyl, indenyl, C₇₋₁₁ polycycloalkyl, tetrahydrofuranyl, furanyl, tetrahydropyranyl, pyranyl, tetrahydrothienyl, thienyl, tetrahydrothiopyranyl, thiopyranyl, C₃₋₆ cycloalkyl, or a C₄₋₆ cycloalkyl containing one or two unsaturated bonds, wherein the cycloalkyl and heterocyclic moieties may be optionally substituted by 1 to 3 methyl groups or one ethyl group;

provided that:

a) when R₆ is hydroxyl, then m is 2; or

b) when R₆ is hydroxyl, then r is 2 to 6; or

c) when R₆ is 2-tetrahydropyranyl, 2-tetrahydrothiopyranyl, 2-tetrahydrofuranyl, or 2-tetrahydrothienyl, then m is 1 or 2; or

d) when R₆ is 2-tetrahydropyranyl, 2-tetrahydrothiopyranyl, 2-tetrahydrofuranyl, or 2-tetrahydrothienyl, then r is 1 to 6;

e) when n is 1 and m is 0, then R₆ is other than H in —(CR₄R₅)ₙO(CR₄R₅)ₘR₆;

X is YR₂, halogen, nitro, NR₄R₅, or formyl amine;

Y is O or S(O)_{m′};

m′ is 0, 1, or 2;

X₂ is O Or NR₈;

X₃ is hydrogen or X;

X₄ is

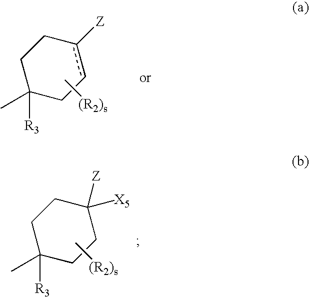

X₅ is H, R₉, OR₈, CN, C(O)R₈, C(O)OR₈, C(O)NR₈R₈, or NR₈R₈;

R₂ is independently selected from-the group consisting of —CH₃ and —CH₂CH₃ optionally substituted by 1 or more halogens;

s is 0 to 4;

R₃ is hydrogen, halogen, C₁₋₄ alkyl, CH₂NHC(O)C(O)NH₂, halo-substituted C₁₋₄ alkyl, —CH═CR_{8′}R_{8′}, cyclopropyl optionally substituted by R_{8′}, CN, OR₈, CH₂OR₈, NR₈R₁₀, CH₂NR₈R₁₀, C(Z′)H, C(O)OR₈, C(O)NR₈R₁₀, or C_CR_{8′};

Z′ is O, NR₉, NOR₈, NCN, C(—CN)₂, CR₈CN, CR₈NO₂, CR₈C(O)OR₈, CR₈C(O)NR₈R₈, C(—CN)NO₂, C(—CN)C(O)OR₉, or C(—CN)C(O)NR₈R₈;

Z is C(Y′)R₁₄, C(O)OR₁₄, C(Y)NR₁₀R₁₄, C(NR₁₀)NR₁₀R₁₄, CN, C(NOR₈)R₁₄, C(O)NR₈NR₈C(O)R₈, C(O)NR₈NR₁₀R₁₄, C(NOR₁₄)R₈, C(NR₈)NR₁₀R₁₄, C(NR₁₄)NR₈R₈, C(NCN)NR₁₀R₁₄, C(NCN)SR₉, (2-, 4- or 5-imidazolyl), (3-, 4- or 5-pyrazolyl), (4- or 5-triazolyl[1,2,3]), (3- or 5-triazolyl[1,2,4]), (5-tetrazolyl), (2-, 4- or 5-oxazolyl), (3-, 4- or 5-isoxazolyl), (3- or 5-oxadiazolyl[1,2,4]), (2-oxadiazolyl[1,3,4]), (2-thiadiazolyl[1,3,4]), (2-, 4-, or 5-thiazolyl), (2-, 4-, or 5-oxazolidinyl), (2-, 4-, or 5-thiazolidinyl), or (2-, 4-, or 5-imidazolidinyl); wherein all of the heterocylic ring systems may be optionally substituted one or more times by R₁₄;

the dotted line in formula (a) represents a single or double bond;

Y′ is O or S;

R₇ is —(CR₄R₅)_{q}R₁₂ or C₁₋₆ alkyl wherein the R₁₂ or C₁₋₆ alkyl group is unsubstituted or substituted one or more times by —F, —Br, —Cl, —NO₂, —NR₁₀R₁₁, —C(O)R₈, —C(O)OR₈, —OR₈, —CN, —C(O)NR₁₀R₁₁, —OC(O)NR₁₀R₁₁, —OC(O)R₈, —NR₁₀C(O)NR₁₀R₁₁, —NR₁₀C(O)R₁₁, —NR₁₀C(O)OR₉, —NR₁₀C(O)R₁₃, —C(NR₁₀)NR₁₀R₁₁, —C(NCN)NR₁₀R₁₁, —C(NCN)SR₉, —NR₁₀C(NCN)SR₉, —NR₁₀C(NCN)NR₁₀R₁₁, —NR₁₀S(O)₂R₉, —S(O)ₘR₉, —NR₁₀C(O)C(O)NR₁₀R₁₁, —NR₁₀C(O)C(O)R₁₀, thiazolyl, imidazolyl, oxazolyl, pyrazolyl, triazolyl, tetrazolyl. C₁₋₂ alkyl optionally substituted by one to three fluorines;

q is 0, 1, or 2;

R₁₂ is C₃₋₇ cycloalkyl, (2-, 3- or 4-pyridyl), pyrimidyl, pyrazolyl, (1- or 2-imidazolyl), thiazolyl, triazolyl, pyrrolyl, piperazinyl, piperidinyl, morpholinyl, furanyl, (2- or 3-thienyl), (4- or 5-thiazolyl), quinolinyl, naphthyl, or phenyl;

R₈ is independently selected from hydrogen or R₉;

R_{8′} is R₈ or fluorine;

R₉ is C₁₋₄ alkyl optionally substituted by one to three fluorines;

R₁₀ is OR₈ or R₁₁;

R₁₁ is hydrogen, or C₁₋₄ alkyl optionally substituted by one to three fluorines; or when R₁₀ and R₁₁ are as NR₁₀R₁₁ they may together with the nitrogen form a 5 to 7 membered ring optionally containing at least one additional heteroatom selected from O, N, or S;

R₁₃ is oxazolidinyl, oxazolyl, thiazolyl, pyrazolyl, triazolyl, tetrazolyl, imidazolyl, inidazolidinyl, thiazolidinyl, isoxazolyl, oxadiazolyl, or thiadiazolyl, and each of these heterocyclic rings is connected through a carbon atom and each may be unsubstituted or substituted by one or two C₁₋₂ alkyl groups;

R₁₄ is hydrogen or R₇; or when R₁₀ and R₁₄ are as NR₁₀R₁₄ they may together with the nitrogen form a 5 to 7 membered ring optionally containing one or more additional heteroatoms selected from 0, N, or S;

or the pharmaceutically acceptable salts thereof.

These compounds are described in U.S. Pat. No. 5,552,438 issued Sep. 3, 1996. It and the compounds it discloses are incorporated herein in full by reference, including the subgeneric preferred groups described therein. Preferred are those compounds of the Formula (I) wherein R₁ is —CH₂-cyclopropyl, —CH₂—C₅₋₆ cycloalkyl, —C₄₋₆ cycloalkyl, tetrahydrofuran-3-yl, (3- or 4-cyclopentenyl), benzyl or —C₁₋₂ alkyl optionally substituted by 1 or more fluorines, and —(CH₂)₂₋₄ OH; R₂ is methyl or fluoro-substituted alkyl, R₃ is CN or C≡CR₈; and X is YR₂. Most preferred are those compounds wherein R₁ is —CH₂-cyclopropyl, cyclopentyl, methyl or CF₂H; R₃ is CN or C≡CH; X is YR₂; Y is oxygen; X₂ is oxygen; X₃ is hydrogen; and R₂ is CF₂H or methyl. The most preferred compound is cis-4-cyano-4-[3-(cyclopentyloxy)-4-methoxyphenyl]cyclohexane-1-carboxylic acid and its salts, esters, pro-drugs and physical forms.

A second perferred group of compounds that can be used in this method is that of Formula (II) or a solvate, hydrate or polymorph thereof, either alone or combined with a pharmaceutically accepteble excipient, wherein Formula (II) comprises:

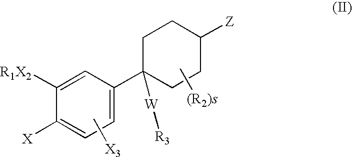

wherein:

R₁ is —(CR₄R₅)ₙC(O)O(CR₄R₅)ₘR₆, —(CR₄R₅)ₙC(O)NR₄(CR₄R₅)ₘR₆, —(CR₄R₅)ₙO(CR₄R₅)ₘR₆, or —(CR₄R₅)ᵣR₆ wherein the alkyl moieties unsubstituted or substituted with one or more halogens;

m is 0 to 2;

n is 0 to 4;

r is 0 to 6;

R₄ and R₅ are independently selected hydrogen or C₁₋₂ alkyl;

R₆ is hydrogen, methyl, hydroxyl,-aryl, halo substituted aryl, aryloxyC₁₋₃ alkyl, halo substituted aryloxyC₁₋₃ alkyl, indanyl, indenyl, C₇₋₁₁ polycycloalkyl, tetrahydrofuranyl, furanyl, tetrahydropyranyl, pyranyl, tetrahydrothienyl, thienyl, tetrahydrothiopyranyl, thiopyranyl, C₃₋₆ cycloalkyl, or a C₄₋₆ cycloalkyl containing one or two unsaturated bonds, wherein the cycloalkyl or heterocyclic moiety is unsubstituted or substituted by 1 to 3 methyl groups, one ethyl group, or an hydroxyl group;

provided that:

a) when R₆ is hydroxyl, then m is 2; or

b) when R₆ is hydroxyl, then r is 2 to 6; or

c) when R₆ is 2-tetrahydropyranyl, 2-tetrahydrothiopyranyl, 2-tetrahydrofuranyl, or 2-tetrahydrothienyl, then m is 1 or 2; or

d) when R₆ is 2-tetrahydropyranyl, 2-tetrahydrothiopyranyl, 2-tetrahydrofuranyl, or 2-tetrahydrothienyl, then r is 1 to 6;

e) when n is 1 and m is 0, then R₆ is other than H in —(CR₄R₅)ₙO(CR₄R₅)ₘR₆;

X is YR₂, fluorine, NR₄R₅, or formyl amine;

Y is O or S(O)_{m′};

m′ is 0, 1, or 2;

X₂ is O Or NR₈;

X₃ is hydrogen or X;

X₄ is H, R₉, OR₈, CN, C(O)R₈, C(O)OR₈, C(O)NR₈R₈, or NR₈R₈;

R₂ is independently selected from —CH₃ or —CH₂CH₃ optionally substituted by 1 or more halogens;

s is 0 to 4;

W is alkyl of 2 to 6 carbons, alkenyl of 2 to 6 carbon atoms or alkynyl of 2 to 6 carbon atoms;

R₃ is COOR₁₄, C(O)NR₄R₁₄ or R₇;

Z is OR₁₄, OR₁₅, SR₁₄, S(O)_{m′}R₇, S(O)₂NR₁₀R₁₄, NR₁₀R₁₄, NR₁₄C(O)R₉, NR₁₀C(Y′)R₁₄, NR₁₀C(O)OR₇, NR₁₀C(Y′)NR₁₀R₁₄, NR₁₀S(O)₂NR₁₀R₁₄, NR₁₀C(NCN)NR₁₀R₁₄, NR₁₀S(O)₂R₇, NR₁₀C(CR₄NO₂)NR₁₀R₁₄, NR₁₀C(NCN)SR₉, NR₁₀C(CR₄NO₂)SR₉, NR₁₀C(NR₁₀)NR₁₀R₁₄, NR₁₀C(O)C(O)NR₁₀R₁₄, or NR₁₀C(O)C(O)OR₁₄;

Y′ is O or S;

R₇ is —(CR₄)_{q}R₁₂ or C₁₋₆ alkyl wherein the R₁₂ or C₁₋₆ alkyl group is unsubstituted or substituted one or more times by methyl or ethyl unsubstituted or substituted by 1-3 fluorines, —F, —Br, —Cl, —NO₂, —NR₁₀R₁₁, —C(O)R₈, —CO₂R₈, —O(CH₂)₂₋₄OR₈, —O(CH₂)_{q}R₈, —CN, —C(O)NR₁₀R₁₁, —O(CH₂)_{q}C(O)NR₁₀R₁₁, —O(CH₂)_{q}C(O)R₉, —NR₁₀C(O)NR₁₀R₁₁, —NR₁₀C(O)R₁₁, —NR₁₀C(O)OR₉, —NR₁₀C(O)R₁₃, —C(NR₁₀)NR₁₀R₁₁, —C(NCN)NR₁₀R₁₁, —C(NCN)SR₉, —NR₁₀C(NCN)SR₉, —NR₁₀C(NCN)NR₁₀R₁₁, —NR₁₀S(O)₂R₉, —S(O)ₘR₉, —NR₁₀C(O)C(O)NR₁₀R₁₁, —NR₁₀C(O)C(O)R₁₀, or R₁₃;

q is 0, 1, or 2;

R₁₂ is R₁₃, C_{3-C}₇ cycloalkyl, or an unsubstituted or substituted aryl or heteroaryl group selected from the group consisting of (2-, 3- or 4-pyridyl), pyrimidinyl, pyrazolyl, (1- or 2-imidazolyl), pyrrolyl, piperazinyl, piperidinyl, morpholinyl, furanyl, (2- or 3-thienyl), quinolinyl, naphthyl, and phenyl;

R₈ is independently selected from hydrogen or R₉;

R₉ is C₁₋₄ alkyl optionally substituted by one to three fluorines;

R₁₀ is OR₈ or R₁₁;

R₁₁ is hydrogen, or C₁₋₄ alkyl unsubstituted or substituted by one to three fluorines; or when R₁₀ and R₁₁ are as NR₁₀R₁₁ they may together with the nitrogen form a 5 to 7 membered ring comprised of carbon or carbon and one or more additional heteroatoms selected from O, N, or S;

R₁₃ is a substituted or unsubstituted heteroaryl group selected from the group consisting of oxazolidinyl, oxazolyl, thiazolyl, pyrazolyl, triazolyl, tetrazolyl, imidazolyl, imidazolidinyl, thiazolidinyl, isoxazolyl, oxadiazolyl, and thiadiazolyl, and where R₁₃ is substituted on R₁₂ or R₁₃ the rings are connected through a carbon atom and each second R₁₃ ring may be unsubstituted or substituted by one or two C₁₋₂ alkyl groups unsubstituted or substituted on the methyl with 1 to 3 fluoro atoms;

R₁₄ is hydrogen or R₇; or when R₈ and R₁₄ are as NR₈R₁₄ they may together with the nitrogen form a 5 to 7 membered ring comprised of carbon or carbon and one or more additional heteroatoms selected from O, N, or S;

R₁₅ is C(O)R₁₄, C(O)NR₈R₁₄, S(O)_{q}NR₈R₁₄ or S(O)_{q}R₇ where q is 0, 1 or 2;

or the pharmaceutically acceptable salts thereof.

Preferred are those compounds of Formula (I) wherein R₁ is —CH₂-cyclopropyl, —CH₂—C₅₋₆ cycloalkyl, —C₄₋₆ cycloalkyl unsubstituted or substituted by OH, tetrahydrofuran-3-yl, (3- or 4-cyclopentenyl), benzyl or —C₁₋₂ alkyl unsubstituted or substituted by 1 or more fluorines, and —(CH₂)₂₋₄ OH; R₂ is methyl or fluoro-substituted alkyl, W is ethynyl or 1,3-butadiynyl; R₃ is R₇ where R₇ is an unsubstituted or substituted aryl or heteroaryl ring, X is YR₂, and Z is OR₁₄, OR₁₅, NR₁₀R₁₄, or NR₁₄C(O)R₉. Most preferred are those compounds wherein R₁ is —CH₂-cyclopropyl, cyclopentyl, 3-hydroxycyclopentyl, methyl or CF₂H; X is YR₂; Y is oxygen; X₂ is oxygen; X₃ is hydrogen; and R₂ is CF₂H or methyl, W is ethynyl or 1,3-butadiynyl, and R₃ is a substituted or unsubstituted pyrimidinyl ring. The most perferred compounds are cis-[4-(2-aminopyrimidin-5-ylethynyl)4-(3-cyclopentyloxy-4-methoxyphenyl)cyclohexan-l-ol] and transA-(2-aminopyrimidin-5-ylethynyl)-4-(3-cyclopentyloxy4-methoxyphenyl)cyclohexan-l-amine and its cyclohexylsulfmate salt.

These compounds and their preparation are described in U.S. Pat. No. 4,981,883 which is incorporated by reference herein in full.

Other compounds of interest include:

AWD-12-281 from Astra (Hofgen, N. et al. 15th EFMC Int Symp Med Chem (September 6-10, Edinburgh) 1998, Abst P.98); a 9-benzyladenine derivative nominated NCS-613 (INSERM); D4418 from Chiroscience and Schering-Plough; a benzodiazepine PDE4 inhibitor identified as CI-1018 (PD-168787; Parke-Davis/Warner-Lambert); a benzodioxole derivative Kyowa Hakko disclosed in WO 9916766; V-11294A from Napp (Landells, L. J. et al. Eur Resp J [Annu Cong Eur Resp Soc (Sept 19-23, Geneva) 1998] 1998, 12(Suppl. 28): Abst P2393); roflumilast (CAS reference No 162401-32-3) and a pthalazinone (WO 9947505) from Byk-Gulden; a compound identified as T-440 (Tanabe Seiyaku; Fuji, K. et al. J Pharmacol Exp Ther, 1998, 284(1): 162), and Bay-19-8004 by Bayer AG, for example.

Dosage Forms

A pharmaceutical composition of the invention is preferably adapted for oral, parenteral or rectal administration. As such it may be in the form of a tablet, capsule, an oral liquid preparation, a powder, granules, a lozenges, a reconstitutable powder, an injectable or infusible solution or suspension or a suppository. An orally administrable compositions are generally preferred.

Tablets and capsules for oral administration may be in unit dose form, and may contain conventional excipients, such as binding agents, fillers, tabletting lubricants, disintegrants and acceptable wetting agents. The tablets may be coated according to methods well known in normal pharmaceutical practice.

Oral liquid preparations may be in the form of, for example, aqueous or oily suspension, solutions, emulsions, syrups or elixirs, or may be in the form of a dry product for reconstitution with water or other suitable vehicle before use. Such liquid preparations may contain conventional additives such as suspending agents, emulsifying agents, non-aqueous vehicles (which may include edible oils), preservatives, and, if desired, conventional flavorings or colorants.

For parenteral administration, fluid unit dosage forms are prepared utilizing a compound of the invention or pharmaceutically acceptable salt thereof and a sterile vehicle. The compound, depending on the vehicle and concentration used, can be either suspended or dissolved in the vehicle. In preparing solutions, the compound can be dissolved for injection and filter sterilized before filling into a suitable vial or ampoule and sealing. Advantageously, adjuvants such as a local anaesthetic, preservatives and buffering agents are dissolved in the vehicle. To enhance the stability, the composition can be frozen after filling into the vial and the water removed under vacuum. Parenteral suspensions are prepared in substantially the same manner, except that the compound is suspended in the vehicle instead of being dissolved, and sterilization cannot be accomplished by filtration. The compound can be sterilized by exposure to ethylene oxide before suspension in a sterile vehicle. Advantageously, a surfactant or wetting agent is included in the composition to facilitate uniform distribution of the compound.

The composition may contain from 0.1% to 99% by weight, preferably from 10 to 60% by weight, of the active material, depending on the method of administration.

The dose of the compound used in the treatment of the aforementioned disorders will vary in the usual way with the seriousness of the disorders, the weight of the sufferer, and other similar factors. However, as a general guide suitable unit doses may be 0.05 to 1000 mg, more suitably 0.05 to 20.0 mg, for example 0.2 to 5 mg; and such unit doses may be administered more than once a day, for example two or three a day, so that the total daily dosage is in the range of about 0.5 to 100 mg; and such therapy may extend for a number of weeks or months.

These compounds can be administered in immediate release form or as an extend or delayed release preparation. At higher dose levels it may be preferred to administer the compound as a controlled release preparation. See copending U.S. application Ser. No. 09/496,799 filed Feb. 2, 2000.

Assays

### EXAMPLE 1

### Phosphodiesterase and Rolipram Binding Assays

### Example 1A

Isolated human monocyte PDE4 and hrPDE4 was determined to exist primarily in the low affinity form. Hence, the activity of test compounds against the low affinity form of PDE 4 can be assessed using standard assays for PDE4 catalytic activity employing 1 μM [³H]cAMP as a substrate (Torphy et al., 1992).

Rat brain high-speed supernatants were used as a source of protein. Enantionmers of [³H]-rolipram were prepared to a specific activity of 25.6 Ci/mmol. Standard assay conditions were modified from the published procedure to be identical to the PDE assay conditions, except for the last of the cAMP: 50 mM Tris HCl (pH 7.5), 5 mM MgCl₂, and 1 nM of [³H]-rolipram (Torphy et al., The J. of Biol. Chem., Vol 267, No. 3, pp 1798-1804, 1992). The assay was run for 1 hour at 30° C. The reaction was terminated and bound ligand was separated from free ligand using a Brandel cell harvester. Competition for the high affinity binding site was assessed under conditions that were identical to those used for measuring low affinity PDE activity, expect that [³H]-cAMP was not present

### Example 1B

Measurement of Phosphodiesterase Activity

PDE activity was assayed using a [³H]cAMP scintillation proximity assay (SPA) or [³HJ]cGMP SPA enzyme assay as described by the supplier (Amersham Life Sciences). The reactions were conducted in 96-well plates at room temperature, in 0.1 ml of reaction buffer containing (final concentrations): 50 mM Tris-HCl, pH 7.5, 8.3 mM MgCl₂, 1.7 mM EGTA, [³H]cAMP or [3H] cGMP (approximately 2000 dpm/pmol), enzyme and various concentrations of the inhibitors. The assay was allowed to proceed for 1 hr and was terminated by adding 50 microliters of SPA yttrium silicate beads in the presence of zinc sulfate. The plates were shaken and allowed to stand at room temperature for 20 min. Radiolabeled product formation was assessed by scintillation spectrometry. Activities of PDE3 and PDE7 were assessed using 0.05 uM [³H]cAMP, whereas PDE4 was assessed using 1 uM [³H]cAMP as a substrate. Activity of PDE1B, PDE1C, PDE2 and PDES activities were assessed using 1 uM [³H]cGMP as a substrate.

[³H]R-Rolipram Binding Assay

The [³H]R-rolipram binding assay was performed by modification of the method of Schneider and co-workers, see Nicholson, et al., Trends Pharmacol. Sci., Vol. 12, pp.19-27 (1991) and McHale et al., Mol Pharmacol., Vol. 39, 109-113 (1991). R-rolipram binds to the catalytic site of PDE4 see Torphy et al., Mol. Pharmacol., Vol. 39, pp. 376-384 (1991). Consequently, competition for [³H]R-rolipram binding provides an independent confirmation of the PDE4 inhibitor potencies of unlabeled competitors. The assay was performed at 30° C. for 1 hr in 0.5 ul buffer containing (final concentrations): 50 mM Tris-HCl, pH 7.5, 5 mM MgCl₂, 0.05% bovine serum albumin, 2 nM [³H]R-rolipram (5.7×104 dpm/pmol) and various concentrations of non-radiolabeled inhibitors. The reaction was stopped by the addition of 2.5 ml of ice-cold reaction buffer (without [³H]-R-rolipram) and rapid vacuum filtration (Brandel Cell Harvester) through Whatman GF/B filters that had been soaked in 0.3% polyethylenimine. The filters were washed with an additional 7.5-ml of cold buffer, dried, and counted via liquid scintillation spectrometry.

The following methods were used to examine representative compounds as disclosed herein for their cognition-enhancing effects.

### EXAMPLE 2

### T Maze TEST

In order to demonstrate cognition enhancement using a T maze rats or mice may be grouped or singly housed, typically in groups of six, in a temperature controlled environment (20° C.±1° C.) and maintained on a 12 hour light dark cycle. Animals are food-deprived for a maximum of 23 out of 24 hours.

The T-maze is constructed from matte black Perspex. The stem is typically 90 cm long with two arms 40 cm in length projecting at right angles to form the “T”. The walls of the maze are 20 cm high.

At the end of each arm is cut a panel into which a small food well is placed. Food pellets are placed into the well remotely or manually. A guillotine door at the base of the “T” stem forms a start box when closed and two similar doors placed at the entrance to each arm to confine the animal within an arm or to restrict access to that arm. The apparatus is housed in a small room containing standard laboratory furniture and computer equipment.

Training is conducted over several weeks. Habituation typically lasts five days during which each animal is placed in the maze for a period of ten minutes each day. Both food wells are filled with food pellets and pellets are also placed throughout the length of both arms and along the stem in order to encourage the animals to explore and enter the goal arms.

Animals are then forced to alternate their arm choices on ten daily trials and during this phase only one food pellet is placed in each food well. On trial one the animal is placed in the start box for approximately 5-10 sec, the guillotine door raised and the animal is allowed to enter either of the goal arms. Having chosen an arm the animal is confined to that arm until the food reward is consumed and is then returned to the start box. On the subsequent trials within the session the animals were forced to choose alternating arms. The guillotine door of the previously visited arm is set to the closed position and the animal is forced to enter the previously unchosen arm. This alternating procedure is repeated until ten trials had been made or until ten minutes had elapsed, whichever is the sooner.

During the final phase the animals are trained to alternate arm choices. On the first trial each animal is placed in the start box and, upon release, is allowed a free choice of arms. Both arms are baited for the first trial. On selection of an arm, the door is closed and the animal is allowed to eat the food reward. The animal is then placed back in the start box and upon release is again allowed a free choice of arms. A correct choice is made if the animal enters the previously unvisited arm. Each animal is typically given a total of ten to 30 trials per day. Training continues until the group achievs an appropriate score over several days.

Following completion of training, delays may be introduced between each trial by retaining the animal in the start box for 0, 10, 20, 30 or 40 sec. From this study the minimum effective delay is chosen for further drug studies. Compound effects on cognition may be demonstrated by improved choice accuracy or reduced choice latency following compound administration, in a range of doses prior to training, during training, once stable performance levels have been reached or prior to the introduction of delay or drug (e.g. scoplomine) induced performance deficits.

### Example 3

### Radial Arm Maze

Animals are typically housed singly with a 12:12 light:dark cycle-and may be kept at approximately 85% of their ad Lib weight. Testing is conducted in an 8-arm radial maze with a central platform and 8 arms (typically 10.5 cm wide and 42 cm long). The top of the maze is usually clear so that the animals can make use of extramaze visual cues. Before each session the maze is wiped with a cleaning solution to help mask odor cues. Entries are scored according a pre-determined definition, e.g., when the animal first puts its nose halfway down an arm. The first entry into each arm is rewarded with a food reward. Re-entries are typically not rewarded. The session lasts until all eight arms have been entered or a specified time has elapsed. Animals are tested in the maze to establish baseline performance for a number of sessions.

The measures consist of arm entries until a choice is repeated (entries to repeat), the number of different arms chosen in the first eight entries (arms in first eight), number of entries until all eight arms were chosen (entries/session) and latency in seconds to enter all eight arms. Variants of this procedure exist which involve the animals visiting baited holes in the floor of the experimental chamber rather than baited arms in a maze.

Compound effects on cognition may be demonstrated by studying the effects of the compound on rates of acquisition, stable baseline performance and interval, drug (typically scopolamine) or brain lesion induced performance deficits.

### EXAMPLE 4

### Water Maze Test

Studies are carried out with rats or mice housed in a controlled environment and maintained on a 12 h light/dark cycle with standard lab chow and water available ad libitum.

Animals are trained in a 200 cm diameter water-filled tank to locate a hidden platform submerged just below the surface of the water. The location of the platform remains constant, but for each trial, the animal is required to swim from one of three different starting locations around the edge of the tank. There are no proximal cues in the tank, so the animal has to use a spatial mapping strategy using the distal cues around the room to navigate to the hidden platform. The pool circumference is arbitrarily marked with four start positions, (N, S, E, W) and divided into four virtual quadrants. The platform (typically a 15 cm Perspex disk) is anchored below the surface, and is therefore invisible to the rat swimming in the water. A video camera is positioned directly above the pool and connected to an image analyser. A PC, calculated measurements of latency, pathlength, number of platform crossings and percent time spent in each quadrant for each trial. Each rat receives 4 consecutive trials on day 1 of training and 6 trials on subsequent days although this may be subject to considerable variation in procedure. Rats also receive transfer tests in which the platform is removed from the pool. At the beginning of each trial the rat is lowered gently feet first into the water, facing the wall at a start position (N, S, E, W) which was predetermined randomly. If the platform is found during the transfer test, the trial is stopped, the recording terminated, and the rat left on the platform for a period. If the platform is not found during this time, the rat is retrieved quickly from the water and placed on the platform. Retention of the learned platform position is assessed with transfer tests carried out after or during training and on subsequent days. Improved cognition is demonstrated by improved escape latencies, reduced swim paths or heading angles and various associated measures. Improved cognition may also be demonstrated by improved transfer test performance, as evidenced by increased percentage time spent in the platform quadrant during the transfer tests, or by associated measures. Compound effects on cognition may be demonstrated by studying the effects of the compound on rates of acquisition or on transfer test performance in normal young or aged rats or mice or in animals whose cognition is impaired by drugs (typically scopolamine) or by lesions to the brain.

### EXAMPLE 5

### Delayed Non-Match or Delayed Match Test

Animals are usually housed in pairs with ad lib access to water and food. Lights were on from 07:00 to 19:00 h. Training is carried out in identical operant chambers contained within sound attenuating boxes. Each animal is assigned to a specific box to ensure consistency of results. Each operant chamber is fitted with two retractable levers, usually situated either side of a food magazine (5.0×6.0 cm) gated by a Perspex flap. The levers are connected to a food dispenser container, which provides the reinforcement. Pellets are dispensed into the food magazine and the animal is required to nose poke the Perspex flap in order to obtain the pellet. A houselight is on throughout the experiment and a second light illuminates the food magazine when in use. A fan to give a constant level of background noise ventilates the box.

Animals are habituated to their assigned operant box during which time food is freely available from the magazine. In further sessions, both the house and magazine lights are switched on and pellets dispensed, to allow association of the magazine with food reinforcement.

Following habituation and magazine training animals progress to a fixed ratio (FR-1) schedule of 30 minutes duration. One of the two levers is randomly extended (even number of presentation for each lever) and illuminated and a pellet is dispensed for every lever response. The number of pellets earned is recorded and animals commence training in the non-matching procedure once they have reached a pre-defined criterion. Each trial begins with the insertion into the operant chamber and illumination of a sample lever (sample stage). The animal is required to press this lever, whereupon the magazine is illuminated. The first nose-poke into the magazine causes the light to be extinguished and both levers to be inserted into the chamber and both lever lights illuminated (choice stage). The animal is required to respond to the lever which is not presented at the sample stage (i.e., make a non-matching response), which results in the delivery of a food pellet and retraction of the levers. An incorrect response or failure to respond at the choice or sample stages during the 20 seconds limited hold (i.e. omission) results in a time-out period of darkness. The next trial is signaled by illumination of the houselight and an inter-trial interval. Training sessions are typically 96 trials or 60 min. A correction procedure is used during this stage of training, such that an incorrect response causes the same lever to be presented in the next trial until the animal responded correctly. Short delay periods of 1-8 seconds between the sample and choice stages are introduced once animals are responding with appropriate accuracy.

Eventually animals are trained using delay periods of typically 0, 2, 4, 8, 16 and 24 seconds. Animals are required to return to the magazine between the sample and choice stages and the first nose-poke made after the delay period had elapsed caused both levers to be presented for the choice stage. The animals are trained until a stable level of performance is reached. Performance measures included percentage correct responses for all completed trials and for individual delays, total number of missed trials (omissions), latencies to respond to the sample lever (sample latency) and retrieve the food pellet (magazine latency) and number of nosepokes/sec during the delay period.

Compound effects on cognition are demonstrated by studying the effects of the compound on rates of acquisition, stable baseline performance and interval, drug (typically scopolamine) or brain lesion induced performance deficits.

### EXAMPLE 6

### Passive Avoidance Procedure

Normal young or aged rats or mice are group housed in environmentally controlled conditions with ad lib access to food and water. Animals are randomly assigned to treatment conditions. The passive avoidance method detects learning, memory and antiamnesic activity. Animals are typically placed individually into the light compartment (usually 30×30×30 cm) of a two-compartment box. After a suitable interval, typically 30 seconds, the door to the dark compartment is opened and when the animal enters the dark compartment, the door is closed and the animal immediately receives a footshock, typically 0.8 mA foot-shock. The latency to cross the dark compartment is recorded. The animal is removed immediately after the shock and replaced in its home cage. After a suitable period (typically 30 min-168 hours) the animal is placed again in the light compartment with the door closed. The door is opened after 30 seconds and the latency to cross to the dark compartment recorded. An increase in latency from Session 1 to Session 2 indicates that the animal has remembered the shock received at Session 1.

Compound effects on cognition may be demonstrated by studying the effects of the compound on rates of acquisition or on recall performance (assessed by latency) using normal young rats or mice or animals in which performance has been degraded by prolonged intervals between training and testing or by drug treatment (typically scopolamine) or brain lesion induced performance deficits.

### EXAMPLE 7

### Five-Choice Tests

Mice or rats, typically male Lister hooded rats (typically housed in pairs in a temperature controlled (21° C.) room under diurnal conditions) are used. Rats are food restricted and maintained at 85% of their free-feeding weight throughout the experiment while water was available ad libitum.

The test apparatus for these experiments consist of 25×25 cm chambers. The rear wall of each chamber is concave and contains 9 apertures, each 2.5 cm square, 4 cm deep and set 2 cm above floor level. Illumination of each hole is provided by a bulb located at the rear of the hole. In addition, each hole has an infra-red photocell beam monitoring the entrance and each hole can be blocked by a metal cover when not required.

Each test chamber is individually housed within sound-attenuating cabinets, ventilated by low-level noise fans, which also serv to mask extraneous background noise. Each chamber is illuminated by a 3W house-light mounted in the centre of the roof. Animals are placed in the chamber through a Perspex door located in the front wall. Directly below this door, animals obtain access to the food magazine by pushing a hinged Perspex panel monitored by a microswitch. Food pellets (45 mg, dustless, Noyes, UK) are dispensed automatically into the magazine. The distance from the magazine panel to each of the holes in the rear wall is 25 cm. The apparatus and on-line data collection is controlled by means of aPC.

Rats are trained to discriminate a brief visual stimulus presented randomly in one of 5 spatial locations, as described previously (Jones et al., 1995a, b). The task contains elements not only of a sustained attention paradigm, the animal being required to monitor the apertures for brief presentations of the visual target during the 30 min session, but also requires the animal to divide attention across the five spatial locations.

The training procedure for this task begins with two 15-min sessions with the response apertures covered with metal caps. During these sessions, the magazine panel is partially open and food pellets placed in the tray. In the next two 30-min sessions, the metal caps are removed from five of the apertures and several food pellets placed within each aperture as well as within the food tray. During the fifth session the test schedule is implemented.

At the beginning of each test session, the house light is illuminated and free delivery of a single food pellet to the magazine made. The trial is initiated by the rat opening the magazine panel to collect this pellet. After a fixed 5 sec inter-trial interval (ITI), the light at the rear of one of the apertures is illuminated for 0.5 sec. Responses in this aperture during illumination and for 5-sec afterwards (the limited hold period) are rewarded with the delivery of a food pellet and a correct response is recorded. Additional responses in the apertures are recorded as perseverative responses and result in a 5 sec period of darkness (time-out). Further responding in the apertures during the time-out restart this period. Responses in a non-illuminated hole during the signal period (incorrect response) and failures to respond within the limited hold period (omission) are similarly punished with a period of darkness. Once again, responses made in an aperture during this period restart the time-out.

A response in the food panel after the delivery of a food pellet, or after the time-out period, initiate the next trial. Additional responses in the panel during the In or time-out periods are recorded but have no further consequences. Responses in the apertures during the ITI are recorded as anticipatory responses and result in a time-out period of darkness, additional responses during this time restart the time-out period. During any one session, the light stimulus is presented an equal number of times in each of the five holes in a random order. A daily session consists of 100 trials or is terminated after 30 minutes of testing. The end of a test session is signalled by extinguishing all the lights. For the first session of training, the stimulus duration and limited hold periods are both set at 1 minute, and the ITI and time-out periods set at 3 seconds. These variables are altered on subsequent trials according to the individual animal's performance, until the target set of task parameters could be instituted. The target parameters were: stimulus duration, 0.5 sec; limited hold period, 5 sec; ITI and time-out period, 5 sec. The animals are considered to have reached criterion when these target parameters are attained on five consecutive sessions with >80% correct responses and <20% omissions within the 30 minute session time. Approximately 30 sessions are required for animals to attain this criterion.

Performance of the task is assessed using the following behavioural measures:

(i) Accuracy. This measures accuracy of responding in a divided attention task where attention is spread over a range of spatial locations. Accuracy of performance is measured as the proportion of responses that are correct (number of correct responses/total number of responses), expressed as a percentage.

(ii) Speed. The latency to respond correctly is defined as the time between the onset of the visual stimulus and the point at which the animal's nose breaks the infra-red beam of the lit hole.

(iii) Errors of Omission. The number of trials on which no response was made during the limited hold period. This measure reflects possible failures of detection and also motivational/motor deficits, depending on the overall pattern of effects.

The effects of test compounds upon performance can be assessed under standard tests conditions (as above), or under a variety of parameter manipulations which impair baseline performance. These include reduced stimulus duration, reduced stimulus brightness, variable inter-stimulus interval, use of a white noise distracter, use of drugs (typically scopolamine), brain lesions or a combination of several parametric manipulations (see Jones et al., 1995, J. Neurosci. 15(11): 7282-7292; (Muir, 1996, Cogn. Brain Res., 3: 215-225).

What is claimed is:

1. A method for enhancing cognitive function by administering to a patient in need thereof an effective amount of a PDE4 inhibitor which has an IC₅₀ ratio of about 0.1 or greater as regards the IC₅₀ for the PDE IV catalytic form which binds rolipram with a high affinity divided by the IC₅₀ for the form which binds rolipram with a low affinity.

2. The method of claim 1 wherein the compound is selected from the group consisting of
cis-4-cyano-4-[3-(cyclopentyloxy)4-methoxyphenyl]cyclohexane-1-carboxylic acid;
cis-4-(2-aminopyrimidin-5-ylethynyl)-4-(3-cyclopentyloxy-4-methoxyphenyl)cyclohexan-1-ol; trans-4-(2-aminopyrimidin-5-ylethynyl)-4-(3-cyclopentyloxy-4-methoxyphenyl)cyclohexan-1-amine or a salt thereof, AWD-12-281; NCS-613; CI-1018; V-11294A; roflumilast; and T-440.

3. A use of a PDE4 inhibitor which has an IC₅₀ ratio of about 0.1 or greater as regards the IC₅₀ for the PDE IV catalytic form which binds rolipram with a high affinity divided by the IC₅₀ for the form which binds rolipram with a low affinity for the manufacture of a medicament for enhancing cognitive function.

4. A use according to claim 1, wherein the compound is selected from the group consisting of
cis-4-cyano-4-[3-(cyclopentyloxy)-4-methoxyphenyl]cyclohexane-1-carboxylic acid;
cis-4-(2-aminopyrimidin-5-ylethynyl)-4-(3-cyclopentyloxy-4-methoxyphenyl)cyclohexan-1-ol; trans-4-(2-aminopyrimidin-5-ylethynyl)-4-(3-cyclopentyloxy-4-methoxyphenyl)cyclohexan-1-amine or a salt thereof, AWD-12-281; NCS-613; CI-1018; V-11294A; roflumilast; and T-440.
